SEQUENCE LISTING (286 sequences)

SEQ ID NO: 1 - DNA, 30 bp, Artificial Sequence
  Probe for L1 gene of HPV6
attatgtgcatccgtaactacatcttccac

SEQ ID NO: 2 - DNA, 30 bp, Artificial Sequence
  Probe for L1 gene of HPV6
ttatgtgcatccgtaactacatcttccaca

SEQ ID NO: 3 - DNA, 30 bp, Artificial Sequence
  Probe for L1 gene of HPV11
aattagtgtatgtagcagatttagacacag

SEQ ID NO: 4 - DNA, 30 bp, Artificial Sequence
  Probe for L1 gene of HPV11
attagtgtatgtagcagatttagacacaga

SEQ ID NO: 5 - DNA, 30 bp, Artificial Sequence
  Probe for L1 gene of HPV11
tagtgtatgtagcagatttagacacagatg

SEQ ID NO: 6 - DNA, 30 bp, Artificial Sequence
  Probe for L1 gene of HPV11
tgtagcagatttagacacagatgcacatag

SEQ ID NO: 7 - DNA, 30 bp, Artificial Sequence
  Probe for L1 gene of HPV11
gtagcagatttagacacagatgcacatagt

SEQ ID NO: 8 - DNA, 30 bp, Artificial Sequence
  Probe for L1 gene of HPV34
agtttgtgtaggtacacaatccacaagtac

SEQ ID NO: 9 - DNA, 30 bp, Artificial Sequence
  Probe for L1 gene of HPV34
gtttgtgtaggtacacaatccacaagtaca

SEQ ID NO: 10 - DNA, 30 bp, Artificial Sequence
  Probe for L1 gene of HPV34
tttgtgtaggtacacaatccacaagtacaa

SEQ ID NO: 11 - DNA, 30 bp, Artificial Sequence
  Probe for L1 gene of HPV34
ttgtgtaggtacacaatccacaagtacaac

SEQ ID NO: 12 - DNA, 30 bp, Artificial Sequence
  Probe for L1 gene of HPV34
tgtgtaggtacacaatccacaagtacaact

SEQ ID NO: 13 - DNA, 30 bp, Artificial Sequence
  Probe for L1 gene of HPV34
gtgtaggtacacaatccacaagtacaactg

SEQ ID NO: 14 - DNA, 30 bp, Artificial Sequence
  Probe for L1 gene of HPV34
tgtaggtacacaatccacaagtacaactgc

SEQ ID NO: 15 - DNA, 30 bp, Artificial Sequence
  Probe for L1 gene of HPV34
gtaggtacacaatccacaagtacaactgca

SEQ ID NO: 16 - DNA, 30 bp, Artificial Sequence
  Probe for L1 gene of HPV34
taggtacacaatccacaagtacaactgcac

SEQ ID NO: 17 - DNA, 30 bp, Artificial Sequence
  Probe for L1 gene of HPV34
agtacaactgcaccatatgcaaacagtaat

SEQ ID NO: 18 - DNA, 30 bp, Artificial Sequence
  Probe for L1 gene of HPV34
aagtacaactgcaccatatgcaaacagtaa

SEQ ID NO: 19 - DNA, 30 bp, Artificial Sequence
  Probe for L1 gene of HPV34
caagtacaactgcaccatatgcaaacagta

SEQ ID NO: 20 - DNA, 30 bp, Artificial Sequence
  Probe for L1 gene of HPV34
acaagtacaactgcaccatatgcaaacagt

SEQ ID NO: 21 - DNA, 30 bp, Artificial Sequence
  Probe for L1 gene of HPV34
cacaagtacaactgcaccatatgcaaacag

SEQ ID NO: 22 - DNA, 30 bp, Artificial Sequence
  Probe for L1 gene of HPV34
ccacaagtacaactgcaccatatgcaaaca

SEQ ID NO: 23 - DNA, 30 bp, Artificial Sequence
  Probe for L1 gene of HPV40
cacacagtcccccacaccaaccccatataa

SEQ ID NO: 24 - DNA, 30 bp, Artificial Sequence
  Probe for L1 gene of HPV40
acacagtcccccacaccaaccccatataat

SEQ ID NO: 25 - DNA, 30 bp, Artificial Sequence
  Probe for L1 gene of HPV40
cacagtcccccacaccaaccccatataata

SEQ ID NO: 26 - DNA, 30 bp, Artificial Sequence
  Probe for L1 gene of HPV40
acagtcccccacaccaaccccatataataa

SEQ ID NO: 27 - DNA, 30 bp, Artificial Sequence
  Probe for L1 gene of HPV42
gccactgcaacatctggtgatacatataca

SEQ ID NO: 28 - DNA, 30 bp, Artificial Sequence
  Probe for L1 gene of HPV42
ccactgcaacatctggtgatacatatacag

SEQ ID NO: 29 - DNA, 30 bp, Artificial Sequence
  Probe for L1 gene of HPV42
cactgcaacatctggtgatacatatacagc

SEQ ID NO: 30 - DNA, 30 bp, Artificial Sequence
  Probe for L1 gene of HPV42
actgcaacatctggtgatacatatacagct

SEQ ID NO: 31 - DNA, 30 bp, Artificial Sequence
  Probe for L1 gene of HPV42
caacatctggtgatacatatacagctgcta

SEQ ID NO: 32 - DNA, 30 bp, Artificial Sequence
  Probe for L1 gene of HPV42
gcaacatctggtgatacatatacagctgct

SEQ ID NO: 33 - DNA, 30 bp, Artificial Sequence
  Probe for L1 gene of HPV42
tgcaacatctggtgatacatatacagctgc

SEQ ID NO: 34 - DNA, 30 bp, Artificial Sequence
  Probe for L1 gene of HPV44
tcccctccgtctacatatactagtgaacaa

SEQ ID NO: 35 - DNA, 30 bp, Artificial Sequence
  Probe for L1 gene of HPV44
gtcccctccgtctacatatactagtgaaca

SEQ ID NO: 36 - DNA, 30 bp, Artificial Sequence
  Probe for L1 gene of HPV44
agtcccctccgtctacatatactagtgaac

SEQ ID NO: 37 - DNA, 30 bp, Artificial Sequence
  Probe for L1 gene of HPV44
cagtcccctccgtctacatatactagtgaa

SEQ ID NO: 38 - DNA, 30 bp, Artificial Sequence
  Probe for L1 gene of HPV44
acagtcccctccgtctacatatactagtga

SEQ ID NO: 39 - DNA, 30 bp, Artificial Sequence
  Probe for L1 gene of HPV72
atacagcttctaattttcgtgagtatcttc

SEQ ID NO: 40 - DNA, 30 bp, Artificial Sequence
  Probe for L1 gene of HPV72
tacagcttctaattttcgtgagtatcttcg

SEQ ID NO: 41 - DNA, 30 bp, Artificial Sequence
  Probe for L1 gene of HPV72
ctgtatattctgatacagaggacgctgtgg

SEQ ID NO: 42 - DNA, 30 bp, Artificial Sequence
  Probe for L1 gene of HPV18
acacagtctcctgtacctgggcaatatgat

SEQ ID NO: 43 - DNA, 30 bp, Artificial Sequence
  Probe for L1 gene of HPV18
cacagtctcctgtacctgggcaatatgatg

SEQ ID NO: 44 - DNA, 30 bp, Artificial Sequence
  Probe for L1 gene of HPV18
acagtctcctgtacctgggcaatatgatgc

SEQ ID NO: 45 - DNA, 30 bp, Artificial Sequence
  Probe for L1 gene of HPV18
agtctcctgtacctgggcaatatgatgcta

SEQ ID NO: 46 - DNA, 30 bp, Artificial Sequence
  Probe for L1 gene of HPV18
gtctcctgtacctgggcaatatgatgctac

SEQ ID NO: 47 - DNA, 30 bp, Artificial Sequence
  Probe for L1 gene of HPV18
tctcctgtacctgggcaatatgatgctacc

SEQ ID NO: 48 - DNA, 30 bp, Artificial Sequence
  Probe for L1 gene of HPV18
gtacctgggcaatatgatgctaccaaattt

SEQ ID NO: 49 - DNA, 30 bp, Artificial Sequence
  Probe for L1 gene of HPV18
tacctgggcaatatgatgctaccaaattta

SEQ ID NO: 50 - DNA, 30 bp, Artificial Sequence
  Probe for L1 gene of HPV18
acctgggcaatatgatgctaccaaatttaa

SEQ ID NO: 51 - DNA, 30 bp, Artificial Sequence
  Probe for L1 gene of HPV26
tctgcagcatctgcatccactccatttaaa

SEQ ID NO: 52 - DNA, 30 bp, Artificial Sequence
  Probe for L1 gene of HPV26
tatctgcagcatctgcatccactccattta

SEQ ID NO: 53 - DNA, 30 bp, Artificial Sequence
  Probe for L1 gene of HPV26
ttatctgcagcatctgcatccactccattt

SEQ ID NO: 54 - DNA, 30 bp, Artificial Sequence
  Probe for L1 gene of HPV26
attatctgcagcatctgcatccactccatt

SEQ ID NO: 55 - DNA, 30 bp, Artificial Sequence
  Probe for L1 gene of HPV30
tgtggataacgtttgtgtggttgcagatat

SEQ ID NO: 56 - DNA, 30 bp, Artificial Sequence
  Probe for L1 gene of HPV30
gtggataacgtttgtgtggttgcagatata

SEQ ID NO: 57 - DNA, 30 bp, Artificial Sequence
  Probe for L1 gene of HPV30
tggataacgtttgtgtggttgcagatatag

SEQ ID NO: 58 - DNA, 30 bp, Artificial Sequence
  Probe for L1 gene of HPV30
ggataacgtttgtgtggttgcagatatagt

SEQ ID NO: 59 - DNA, 30 bp, Artificial Sequence
  Probe for L1 gene of HPV30
gataacgtttgtgtggttgcagatatagtc

SEQ ID NO: 60 - DNA, 30 bp, Artificial Sequence
  Probe for L1 gene of HPV31
gctgcaattgcaaacagtgatactacattt

SEQ ID NO: 61 - DNA, 30 bp, Artificial Sequence
  Probe for L1 gene of HPV31
tgctgcaattgcaaacagtgatactacatt

SEQ ID NO: 62 - DNA, 30 bp, Artificial Sequence
  Probe for L1 gene of HPV31
gtgctgcaattgcaaacagtgatactacat

SEQ ID NO: 63 - DNA, 30 bp, Artificial Sequence
  Probe for L1 gene of HPV31
ttgtgctgcaattgcaaacagtgatactac

SEQ ID NO: 64 - DNA, 30 bp, Artificial Sequence
  Probe for L1 gene of HPV31
tttgtgctgcaattgcaaacagtgatacta

SEQ ID NO: 65 - DNA, 30 bp, Artificial Sequence
  Probe for L1 gene of HPV31
gtttgtgctgcaattgcaaacagtgatact

SEQ ID NO: 66 - DNA, 30 bp, Artificial Sequence
  Probe for L1 gene of HPV33
actttatgcacacaagtaactagtgacagt

SEQ ID NO: 67 - DNA, 30 bp, Artificial Sequence
  Probe for L1 gene of HPV33
ctttatgcacacaagtaactagtgacagta

SEQ ID NO: 68 - DNA, 30 bp, Artificial Sequence
  Probe for L1 gene of HPV33
actgtcactagttacttgtgtgcataaagt

SEQ ID NO: 69 - DNA, 30 bp, Artificial Sequence
  Probe for L1 gene of HPV33
tactgtcactagttacttgtgtgcataaag

SEQ ID NO: 70 - DNA, 30 bp, Artificial Sequence
  Probe for L1 gene of HPV39
tatctacctctatagagtcttccatacctt

SEQ ID NO: 71 - DNA, 30 bp, Artificial Sequence
  Probe for L1 gene of HPV39
atctacctctatagagtcttccataccttc

SEQ ID NO: 72 - DNA, 30 bp, Artificial Sequence
  Probe for L1 gene of HPV39
cttccataccttctacatatgatccttcta

SEQ ID NO: 73 - DNA, 30 bp, Artificial Sequence
  Probe for L1 gene of HPV39
tcttccataccttctacatatgatccttct

SEQ ID NO: 74 - DNA, 30 bp, Artificial Sequence
  Probe for L1 gene of HPV39
gtcttccataccttctacatatgatccttc

SEQ ID NO: 75 - DNA, 30 bp, Artificial Sequence
  Probe for L1 gene of HPV39
cctctatagagtcttccataccttctacat

SEQ ID NO: 76 - DNA, 30 bp, Artificial Sequence
  Probe for L1 gene of HPV39
acctctatagagtcttccataccttctaca

SEQ ID NO: 77 - DNA, 30 bp, Artificial Sequence
  Probe for L1 gene of HPV45
ctctacacaaaatcctgtgccaagtacata

SEQ ID NO: 78 - DNA, 30 bp, Artificial Sequence
  Probe for L1 gene of HPV45
tctacacaaaatcctgtgccaagtacatat

SEQ ID NO: 79 - DNA, 30 bp, Artificial Sequence
  Probe for L1 gene of HPV45
ctacacaaaatcctgtgccaagtacatatg

SEQ ID NO: 80 - DNA, 30 bp, Artificial Sequence
  Probe for L1 gene of HPV80
tacacaaaatcctgtgccaagtacatatga

SEQ ID NO: 81 - DNA, 30 bp, Artificial Sequence
  Probe for L1 gene of HPV51
tgcggtttccccaacatttactccaagtaa

SEQ ID NO: 82 - DNA, 30 bp, Artificial Sequence
  Probe for L1 gene of HPV51
ctgcggtttccccaacatttactccaagta

SEQ ID NO: 83 - DNA, 30 bp, Artificial Sequence
  Probe for L1 gene of HPV51
gctgcggtttccccaacatttactccaagt

SEQ ID NO: 84 - DNA, 30 bp, Artificial Sequence
  Probe for L1 gene of HPV51
tgctgcggtttccccaacatttactccaag

SEQ ID NO: 85 - DNA, 30 bp, Artificial Sequence
  Probe for L1 gene of HPV51
ctgctgcggtttccccaacatttactccaa

SEQ ID NO: 86 - DNA, 30 bp, Artificial Sequence
  Probe for L1 gene of HPV51
actgctgcggtttccccaacatttactcca

SEQ ID NO: 87 - DNA, 30 bp, Artificial Sequence
  Probe for L1 gene of HPV51
cactgctgcggtttccccaacatttactcc

SEQ ID NO: 88 - DNA, 30 bp, Artificial Sequence
  Probe for L1 gene of HPV51
ccactgctgcggtttccccaacatttactc

SEQ ID NO: 89 - DNA, 30 bp, Artificial Sequence
  Probe for L1 gene of HPV52
tttatgtgctgaggttaaaaaggaaagcac

SEQ ID NO: 90 - DNA, 30 bp, Artificial Sequence
  Probe for L1 gene of HPV52
gtgctgaggttaaaaaggaaagcacatata

SEQ ID NO: 91 - DNA, 30 bp, Artificial Sequence
  Probe for L1 gene of HPV52
gttaaaaaggaaagcacatataaaaatgaa

SEQ ID NO: 92 - DNA, 30 bp, Artificial Sequence
  Probe for L1 gene of HPV52
ggttaaaaaggaaagcacatataaaaatga

SEQ ID NO: 93 - DNA, 30 bp, Artificial Sequence
  Probe for L1 gene of HPV54
gtcagaattattaaagctatcctgcgtgga

SEQ ID NO: 94 - DNA, 30 bp, Artificial Sequence
  Probe for L1 gene of HPV54
tcagaattattaaagctatcctgcgtggat

SEQ ID NO: 95 - DNA, 30 bp, Artificial Sequence
  Probe for L1 gene of HPV54
cagaattattaaagctatcctgcgtggatg

SEQ ID NO: 96 - DNA, 30 bp, Artificial Sequence
  Probe for L1 gene of HPV54
agaattattaaagctatcctgcgtggatgc

SEQ ID NO: 97 - DNA, 30 bp, Artificial Sequence
  Probe for L1 gene of HPV54
gaattattaaagctatcctgcgtggatgct

SEQ ID NO: 98 - DNA, 30 bp, Artificial Sequence
  Probe for L1 gene of HPV54
aattattaaagctatcctgcgtggatgctg

SEQ ID NO: 99 - DNA, 30 bp, Artificial Sequence
  Probe for L1 gene of HPV54
attattaaagctatcctgcgtggatgctgt

SEQ ID NO: 100 - DNA, 30 bp, Artificial Sequence
  Probe for L1 gene of HPV54
ttattaaagctatcctgcgtggatgctgta

SEQ ID NO: 101 - DNA, 30 bp, Artificial Sequence
  Probe for L1 gene of HPV54
tattaaagctatcctgcgtggatgctgtag

SEQ ID NO: 102 - DNA, 30 bp, Artificial Sequence
  Probe for L1 gene of HPV54
attaaagctatcctgcgtggatgctgtagc

SEQ ID NO: 103 - DNA, 30 bp, Artificial Sequence
  Probe for L1 gene of HPV54
ttaaagctatcctgcgtggatgctgtagca

SEQ ID NO: 104 - DNA, 30 bp, Artificial Sequence
  Probe for L1 gene of HPV54
taaagctatcctgcgtggatgctgtagcac

SEQ ID NO: 105 - DNA, 30 bp, Artificial Sequence
  Probe for L1 gene of HPV54
aaagctatcctgcgtggatgctgtagcaca

SEQ ID NO: 106 - DNA, 30 bp, Artificial Sequence
  Probe for L1 gene of HPV54
aagctatcctgcgtggatgctgtagcacac

SEQ ID NO: 107 - DNA, 30 bp, Artificial Sequence
  Probe for L1 gene of HPV54
gctatcctgcgtggatgctgtagcacacaa

SEQ ID NO: 108 - DNA, 30 bp, Artificial Sequence
  Probe for L1 gene of HPV54
ctatcctgcgtggatgctgtagcacacaat

SEQ ID NO: 109 - DNA, 30 bp, Artificial Sequence
  Probe for L1 gene of HPV59
tctattcctaatgtatacacacctaccagt

SEQ ID NO: 110 - DNA, 30 bp, Artificial Sequence
  Probe for L1 gene of HPV59
cttctattcctaatgtatacacacctacca

SEQ ID NO: 111 - DNA, 30 bp, Artificial Sequence
  Probe for L1 gene of HPV59
tcttctattcctaatgtatacacacctacc

SEQ ID NO: 112 - DNA, 30 bp, Artificial Sequence
  Probe for L1 gene of HPV69
tgtatctgcacaatctgcatctgccacttt

SEQ ID NO: 113 - DNA, 30 bp, Artificial Sequence
  Probe for L1 gene of HPV69
aaagtggcagatgcagattgtgcagataca

SEQ ID NO: 114 - DNA, 30 bp, Artificial Sequence
  Probe for L1 gene of HPV70
ctgcaccgaaacggccatacctgctgtata

SEQ ID NO: 115 - DNA, 30 bp, Artificial Sequence
  Probe for L1 gene of HPV70
tgcaccgaaacggccatacctgctgtatat

SEQ ID NO: 116 - DNA, 30 bp, Artificial Sequence
  Probe for L1 gene of HPV70
gcaccgaaacggccatacctgctgtatata

SEQ ID NO: 117 - DNA, 30 bp, Artificial Sequence
  Probe for L1 gene of HPV70
caccgaaacggccatacctgctgtatatag

SEQ ID NO: 118 - DNA, 30 bp, Artificial Sequence
  Probe for L1 gene of HPV70
ccgaaacggccatacctgctgtatatagcc

SEQ ID NO: 119 - DNA, 30 bp, Artificial Sequence
  Probe for L1 gene of HPV70
cgaaacggccatacctgctgtatatagccc

SEQ ID NO: 120 - DNA, 30 bp, Artificial Sequence
  Probe for L1 gene of HPV70
gaaacggccatacctgctgtatatagccct

SEQ ID NO: 121 - DNA, 30 bp, Artificial Sequence
  Probe for L1 gene of HPV70
aaacggccatacctgctgtatatagcccta

SEQ ID NO: 122 - DNA, 30 bp, Artificial Sequence
  Probe for L1 gene of HPV70
acggccatacctgctgtatatagccctaca

SEQ ID NO: 123 - DNA, 30 bp, Artificial Sequence
  Probe for L1 gene of HPV70
cggccatacctgctgtatatagccctacaa

SEQ ID NO: 124 - DNA, 30 bp, Artificial Sequence
  Probe for L1 gene of HPV70
ggccatacctgctgtatatagccctacaaa

SEQ ID NO: 125 - DNA, 30 bp, Artificial Sequence
  Probe for L1 gene of HPV70
gccatacctgctgtatatagccctacaaag

SEQ ID NO: 126 - DNA, 30 bp, Artificial Sequence
  Probe for L1 gene of HPV70
ccatacctgctgtatatagccctacaaagt

SEQ ID NO: 127 - DNA, 30 bp, Artificial Sequence
  Probe for L1 gene of HPV70
catacctgctgtatatagccctacaaagtt

SEQ ID NO: 128 - DNA, 30 bp, Artificial Sequence
  Probe for L1 gene of HPV70
atacctgctgtatatagccctacaaagttt

SEQ ID NO: 129 - DNA, 30 bp, Artificial Sequence
  Probe for L1 gene of HPV70
tacctgctgtatatagccctacaaagttta

SEQ ID NO: 130 - DNA, 30 bp, Artificial Sequence
  Probe for L1 gene of HPV70
acctgctgtatatagccctacaaagtttaa

SEQ ID NO: 131 - DNA, 30 bp, Artificial Sequence
  Probe for L1 gene of HPV70
cctgctgtatatagccctacaaagtttaag

SEQ ID NO: 132 - DNA, 30 bp, Artificial Sequence
  Probe for L1 gene of HPV82
cattagcactgctgttactccatctgttgc

SEQ ID NO: 133 - DNA, 30 bp, Artificial Sequence
  Probe for L1 gene of HPV82
attagcactgctgttactccatctgttgca

SEQ ID NO: 134 - DNA, 30 bp, Artificial Sequence
  Probe for L1 gene of HPV82
ttagcactgctgttactccatctgttgcac

SEQ ID NO: 135 - DNA, 30 bp, Artificial Sequence
  Probe for L1 gene of HPV82
tagcactgctgttactccatctgttgcaca

SEQ ID NO: 136 - DNA, 30 bp, Artificial Sequence
  Probe for L1 gene of HPV82
agcactgctgttactccatctgttgcacaa

SEQ ID NO: 137 - DNA, 30 bp, Artificial Sequence
  Probe for L1 gene of HPV82
gcactgctgttactccatctgttgcacaaa

SEQ ID NO: 138 - DNA, 30 bp, Artificial Sequence
  Probe for L1 gene of HPV82
cactgctgttactccatctgttgcacaaac

SEQ ID NO: 139 - DNA, 30 bp, Artificial Sequence
  Probe for L1 gene of HPV82
actgctgttactccatctgttgcacaaaca

SEQ ID NO: 140 - DNA, 30 bp, Artificial Sequence
  Probe for L1 gene of HPV82
ctgctgttactccatctgttgcacaaacat

SEQ ID NO: 141 - DNA, 30 bp, Artificial Sequence
  Probe for L1 gene of HPV82
tgctgttactccatctgttgcacaaacatt

SEQ ID NO: 142 - DNA, 30 bp, Artificial Sequence
  Probe for L1 gene of HPV82
gctgttactccatctgttgcacaaacattt

SEQ ID NO: 143 - DNA, 30 bp, Artificial Sequence
  Probe for L1 gene of HPV82
ctgttactccatctgttgcacaaacattta

SEQ ID NO: 144 - DNA, 30 bp, Artificial Sequence
  Probe for L1 gene of HPV82
tgttactccatctgttgcacaaacatttac

SEQ ID NO: 145 - DNA, 30 bp, Artificial Sequence
  Probe for L1 gene of HPV82
gttactccatctgttgcacaaacatttact

SEQ ID NO: 146 - DNA, 30 bp, Artificial Sequence
  Probe for L1 gene of HPV82
ttactccatctgttgcacaaacatttactc

SEQ ID NO: 147 - DNA, 30 bp, Artificial Sequence
  Probe for L1 gene of HPV82
tactccatctgttgcacaaacatttactcc

SEQ ID NO: 148 - DNA, 30 bp, Artificial Sequence
  Probe for L1 gene of HPV82
ctccatctgttgcacaaacatttactccag

SEQ ID NO: 149 - DNA, 30 bp, Artificial Sequence
  Probe for L1 gene of HPV82
atctgttgcacaaacatttactccagcaaa

SEQ ID NO: 150 - DNA, 30 bp, Artificial Sequence
  Probe for L1 gene of HPV53
tgtagacatagactgtgtggttgcggaaag

SEQ ID NO: 151 - DNA, 30 bp, Artificial Sequence
  Probe for L1 gene of HPV53
gtagacatagactgtgtggttgcggaaaga

SEQ ID NO: 152 - DNA, 30 bp, Artificial Sequence
  Probe for L1 gene of HPV53
cacagtctatgtctacatataattcaaagc

SEQ ID NO: 153 - DNA, 30 bp, Artificial Sequence
  Probe for L1 gene of HPV53
acagtctatgtctacatataattcaaagca

SEQ ID NO: 154 - DNA, 30 bp, Artificial Sequence
  Probe for L1 gene of HPV53
cagtctatgtctacatataattcaaagcaa

SEQ ID NO: 155 - DNA, 33 bp, Artificial Sequence
  Probe for L1 gene of HPV53
cacagtctatgtctacatataattcaaagcaaa

SEQ ID NO: 156 - DNA, 30 bp, Artificial Sequence
  Probe for L1 gene of HPV61
ccctgtatctgaatataaagccacaagctt

SEQ ID NO: 157 - DNA, 30 bp, Artificial Sequence
  Probe for L1 gene of HPV61
gtatctgaatataaagccacaagctttagg

SEQ ID NO: 158 - DNA, 30 bp, Artificial Sequence
  Probe for L1 gene of HPV61
tatctgaatataaagccacaagctttaggg

SEQ ID NO: 159 - DNA, 30 bp, Artificial Sequence
  Probe for L1 gene of HPV61
atctgaatataaagccacaagctttaggga

SEQ ID NO: 160 - DNA, 30 bp, Artificial Sequence
  Probe for L1 gene of HPV61
attccctaaagcttgtggctttatattcag

SEQ ID NO: 161 - DNA, 30 bp, Artificial Sequence
  Probe for L1 gene of HPV61
ttccctaaagcttgtggctttatattcaga

SEQ ID NO: 162 - DNA, 30 bp, Artificial Sequence
  Probe for L1 gene of HPV61
tccctaaagcttgtggctttatattcagat

SEQ ID NO: 163 - DNA, 30 bp, Artificial Sequence
  Probe for L1 gene of HPV61
ccctaaagcttgtggctttatattcagata

SEQ ID NO: 164 - DNA, 30 bp, Artificial Sequence
  Probe for L1 gene of HPV61
cctaaagcttgtggctttatattcagatac

SEQ ID NO: 165 - DNA, 30 bp, Artificial Sequence
  Probe for L1 gene of HPV61
aaagcttgtggctttatattcagatacagg

SEQ ID NO: 166 - DNA, 30 bp, Artificial Sequence
  Probe for L1 gene of HPV61
aagcttgtggctttatattcagatacaggg

SEQ ID NO: 167 - DNA, 30 bp, Artificial Sequence
  Probe for L1 gene of HPV62
tatttgtaccgcctccactgctgcagcaga

SEQ ID NO: 168 - DNA, 30 bp, Artificial Sequence
  Probe for L1 gene of HPV62
tttgtaccgcctccactgctgcagcagaat

SEQ ID NO: 169 - DNA, 30 bp, Artificial Sequence
  Probe for L1 gene of HPV62
ttgtaccgcctccactgctgcagcagaata

SEQ ID NO: 170 - DNA, 30 bp, Artificial Sequence
  Probe for L1 gene of HPV62
gtaccgcctccactgctgcagcagaataca

SEQ ID NO: 171 - DNA, 30 bp, Artificial Sequence
  Probe for L1 gene of HPV62
ctccactgctgcagcagaatacacggctac

SEQ ID NO: 172 - DNA, 30 bp, Artificial Sequence
  Probe for L1 gene of HPV62
tccactgctgcagcagaatacacggctacc

SEQ ID NO: 173 - DNA, 30 bp, Artificial Sequence
  Probe for L1 gene of HPV62
cactgctgcagcagaatacacggctaccaa

SEQ ID NO: 174 - DNA, 30 bp, Artificial Sequence
  Probe for L1 gene of HPV62
actgctgcagcagaatacacggctaccaac

SEQ ID NO: 175 - DNA, 30 bp, Artificial Sequence
  Probe for L1 gene of HPV62
cctaaagttggtagccgtgtattctgctgc

SEQ ID NO: 176 - DNA, 30 bp, Artificial Sequence
  Probe for L1 gene of HPV62
aagttggtagccgtgtattctgctgcagca

SEQ ID NO: 177 - DNA, 30 bp, Artificial Sequence
  Probe for L1 gene of HPV62
ggtagccgtgtattctgctgcagcagtgga

SEQ ID NO: 178 - DNA, 30 bp, Artificial Sequence
  Probe for L1 gene of HPV62
gtagccgtgtattctgctgcagcagtggag

SEQ ID NO: 179 - DNA, 30 bp, Artificial Sequence
  Probe for L1 gene of HPV67
tttatgttctgaggaaaaatcagaggctac

SEQ ID NO: 180 - DNA, 30 bp, Artificial Sequence
  Probe for L1 gene of HPV67
ttatgttctgaggaaaaatcagaggctaca

SEQ ID NO: 181 - DNA, 30 bp, Artificial Sequence
  Probe for L1 gene of HPV67
atgttctgaggaaaaatcagaggctacata

SEQ ID NO: 182 - DNA, 30 bp, Artificial Sequence
  Probe for L1 gene of HPV67
tgttctgaggaaaaatcagaggctacatac

SEQ ID NO: 183 - DNA, 30 bp, Artificial Sequence
  Probe for L1 gene of HPV67
gttctgaggaaaaatcagaggctacataca

SEQ ID NO: 184 - DNA, 30 bp, Artificial Sequence
  Probe for L1 gene of HPV67
ttctgaggaaaaatcagaggctacatacaa

SEQ ID NO: 185 - DNA, 30 bp, Artificial Sequence
  Probe for L1 gene of HPV67
tctgaggaaaaatcagaggctacatacaaa

SEQ ID NO: 186 - DNA, 30 bp, Artificial Sequence
  Probe for L1 gene of HPV67
ctgaggaaaaatcagaggctacatacaaaa

SEQ ID NO: 187 - DNA, 30 bp, Artificial Sequence
  Probe for L1 gene of HPV67
ggaaaaatcagaggctacatacaaaaatga

SEQ ID NO: 188 - DNA, 30 bp, Artificial Sequence
  Probe for L1 gene of HPV71
catctgtgctaccaaaactgttgagtctac

SEQ ID NO: 189 - DNA, 30 bp, Artificial Sequence
  Probe for L1 gene of HPV71
atctgtgctaccaaaactgttgagtctaca

SEQ ID NO: 190 - DNA, 30 bp, Artificial Sequence
  Probe for L1 gene of HPV71
tctgtgctaccaaaactgttgagtctacat

SEQ ID NO: 191 - DNA, 30 bp, Artificial Sequence
  Probe for L1 gene of HPV71
ctgtgctaccaaaactgttgagtctacata

SEQ ID NO: 192 - DNA, 30 bp, Artificial Sequence
  Probe for L1 gene of HPV71
tgtgctaccaaaactgttgagtctacatat

SEQ ID NO: 193 - DNA, 30 bp, Artificial Sequence
  Probe for L1 gene of HPV71
gtgctaccaaaactgttgagtctacatata

SEQ ID NO: 194 - DNA, 30 bp, Artificial Sequence
  Probe for L1 gene of HPV71
tgctaccaaaactgttgagtctacatataa

SEQ ID NO: 195 - DNA, 30 bp, Artificial Sequence
  Probe for L1 gene of HPV71
accaaaactgttgagtctacatataaagcc

SEQ ID NO: 196 - DNA, 30 bp, Artificial Sequence
  Probe for L1 gene of HPV71
ccaaaactgttgagtctacatataaagcct

SEQ ID NO: 197 - DNA, 30 bp, Artificial Sequence
  Probe for L1 gene of HPV71
caaaactgttgagtctacatataaagcctc

SEQ ID NO: 198 - DNA, 30 bp, Artificial Sequence
  Probe for L1 gene of HPV71
aaaactgttgagtctacatataaagcctct

SEQ ID NO: 199 - DNA, 30 bp, Artificial Sequence
  Probe for L1 gene of HPV71
aaactgttgagtctacatataaagcctcta

SEQ ID NO: 200 - DNA, 30 bp, Artificial Sequence
  Probe for L1 gene of HPV71
aactgttgagtctacatataaagcctctag

SEQ ID NO: 201 - DNA, 30 bp, Artificial Sequence
  Probe for L1 gene of HPV74
tgtgtgtgctcctacctcacaatcgccttc

SEQ ID NO: 202 - DNA, 30 bp, Artificial Sequence
  Probe for L1 gene of HPV74
gtgtgtgctcctacctcacaatcgccttct

SEQ ID NO: 203 - DNA, 30 bp, Artificial Sequence
  Probe for L1 gene of HPV74
tgtgtgctcctacctcacaatcgccttctg

SEQ ID NO: 204 - DNA, 30 bp, Artificial Sequence
  Probe for L1 gene of HPV74
gtgtgctcctacctcacaatcgccttctgc

SEQ ID NO: 205 - DNA, 30 bp, Artificial Sequence
  Probe for L1 gene of HPV74
tgtgctcctacctcacaatcgccttctgct

SEQ ID NO: 206 - DNA, 30 bp, Artificial Sequence
  Probe for L1 gene of HPV74
gtgctcctacctcacaatcgccttctgcta

SEQ ID NO: 207 - DNA, 30 bp, Artificial Sequence
  Probe for L1 gene of HPV74
tgctcctacctcacaatcgccttctgctac

SEQ ID NO: 208 - DNA, 30 bp, Artificial Sequence
  Probe for L1 gene of HPV74
gctcctacctcacaatcgccttctgctaca

SEQ ID NO: 209 - DNA, 30 bp, Artificial Sequence
  Probe for L1 gene of HPV83
gctgctgctacacaggctaatgaatacaca

SEQ ID NO: 210 - DNA, 30 bp, Artificial Sequence
  Probe for L1 gene of HPV83
ctgctgctacacaggctaatgaatacacag

SEQ ID NO: 211 - DNA, 30 bp, Artificial Sequence
  Probe for L1 gene of HPV83
tgctgctacacaggctaatgaatacacagc

SEQ ID NO: 212 - DNA, 30 bp, Artificial Sequence
  Probe for L1 gene of HPV83
gctgctacacaggctaatgaatacacagcc

SEQ ID NO: 213 - DNA, 30 bp, Artificial Sequence
  Probe for L1 gene of HPV83
ctgctacacaggctaatgaatacacagcct

SEQ ID NO: 214 - DNA, 30 bp, Artificial Sequence
  Probe for L1 gene of HPV83
tgctacacaggctaatgaatacacagcctc

SEQ ID NO: 215 - DNA, 30 bp, Artificial Sequence
  Probe for L1 gene of HPV83
gctacacaggctaatgaatacacagcctct

SEQ ID NO: 216 - DNA, 30 bp, Artificial Sequence
  Probe for L1 gene of HPV83
ctacacaggctaatgaatacacagcctcta

SEQ ID NO: 217 - DNA, 30 bp, Artificial Sequence
  Probe for L1 gene of HPV84
tattagtgctgctaccaacaccgaatcaga

SEQ ID NO: 218 - DNA, 30 bp, Artificial Sequence
  Probe for L1 gene of HPV84
attagtgctgctaccaacaccgaatcagaa

SEQ ID NO: 219 - DNA, 30 bp, Artificial Sequence
  Probe for L1 gene of HPV84
ttagtgctgctaccaacaccgaatcagaat

SEQ ID NO: 220 - DNA, 30 bp, Artificial Sequence
  Probe for L1 gene of HPV84
tagtgctgctaccaacaccgaatcagaata

SEQ ID NO: 221 - DNA, 30 bp, Artificial Sequence
  Probe for L1 gene of HPV84
agtgctgctaccaacaccgaatcagaatat

SEQ ID NO: 222 - DNA, 30 bp, Artificial Sequence
  Probe for L1 gene of HPV84
gtgctgctaccaacaccgaatcagaatata

SEQ ID NO: 223 - DNA, 30 bp, Artificial Sequence
  Probe for L1 gene of HPV84
tgctgctaccaacaccgaatcagaatataa

SEQ ID NO: 224 - DNA, 30 bp, Artificial Sequence
  Probe for L1 gene of HPV84
gctgctaccaacaccgaatcagaatataaa

SEQ ID NO: 225 - DNA, 30 bp, Artificial Sequence
  Probe for L1 gene of HPV85
cttatctactgcaactactaatccagttcc

SEQ ID NO: 226 - DNA, 30 bp, Artificial Sequence
  Probe for L1 gene of HPV85
ttatctactgcaactactaatccagttcca

SEQ ID NO: 227 - DNA, 30 bp, Artificial Sequence
  Probe for L1 gene of HPV85
tatctactgcaactactaatccagttccat

SEQ ID NO: 228 - DNA, 30 bp, Artificial Sequence
  Probe for L1 gene of HPV85
atctactgcaactactaatccagttccatc

SEQ ID NO: 229 - DNA, 30 bp, Artificial Sequence
  Probe for L1 gene of HPV85
tctactgcaactactaatccagttccatct

SEQ ID NO: 230 - DNA, 30 bp, Artificial Sequence
  Probe for L1 gene of HPV85
ctactgcaactactaatccagttccatcta

SEQ ID NO: 231 - DNA, 30 bp, Artificial Sequence
  Probe for L1 gene of HPV85
tactgcaactactaatccagttccatctat

SEQ ID NO: 232 - DNA, 30 bp, Artificial Sequence
  Probe for L1 gene of HPV85
actgcaactactaatccagttccatctata

SEQ ID NO: 233 - DNA, 30 bp, Artificial Sequence
  Probe for L1 gene of HPV85
ctgcaactactaatccagttccatctatat

SEQ ID NO: 234 - DNA, 30 bp, Artificial Sequence
  Probe for L1 gene of HPV89
tgcttcccagtctggcacagaatacagttc

SEQ ID NO: 235 - DNA, 30 bp, Artificial Sequence
  Probe for L1 gene of HPV89
gcttcccagtctggcacagaatacagttct

SEQ ID NO: 236 - DNA, 30 bp, Artificial Sequence
  Probe for L1 gene of HPV89
cttcccagtctggcacagaatacagttcta

SEQ ID NO: 237 - DNA, 30 bp, Artificial Sequence
  Probe for L1 gene of HPV89
ttcccagtctggcacagaatacagttctac

SEQ ID NO: 238 - DNA, 30 bp, Artificial Sequence
  Probe for L1 gene of HPV89
tcccagtctggcacagaatacagttctaca

SEQ ID NO: 239 - DNA, 30 bp, Artificial Sequence
  Probe for L1 gene of HPV89
cccagtctggcacagaatacagttctacac

SEQ ID NO: 240 - DNA, 30 bp, Artificial Sequence
  Probe for L1 gene of HPV89
ccagtctggcacagaatacagttctacacg

SEQ ID NO: 241 - DNA, 30 bp, Artificial Sequence
  Probe for L1 gene of HPV89
cagtctggcacagaatacagttctacacgc

SEQ ID NO: 242 - DNA, 30 bp, Artificial Sequence
  Probe for L1 gene of HPV89
agtctggcacagaatacagttctacacgct

SEQ ID NO: 243 - DNA, 30 bp, Artificial Sequence
  Probe for L1 gene of HPV89
gtctggcacagaatacagttctacacgctt

SEQ ID NO: 244 - DNA, 30 bp, Artificial Sequence
  Probe for L1 gene of HPV89
tctggcacagaatacagttctacacgcttt

SEQ ID NO: 245 - DNA, 30 bp, Artificial Sequence
  Probe for L1 gene of HPV89
ctggcacagaatacagttctacacgcttta

SEQ ID NO: 246 - DNA, 30 bp, Artificial Sequence
  Probe for L1 gene of HPV89
tggcacagaatacagttctacacgctttaa

SEQ ID NO: 247 - DNA, 30 bp, Artificial Sequence
  Probe for L1 gene of HPV89
ggcacagaatacagttctacacgctttaag

SEQ ID NO: 248 - DNA, 30 bp, Artificial Sequence
  Probe for L1 gene of HPV90
gccacacaaacaccctctgacacatacaag

SEQ ID NO: 249 - DNA, 30 bp, Artificial Sequence
  Probe for L1 gene of HPV90
ccacacaaacaccctctgacacatacaagg

SEQ ID NO: 250 - DNA, 30 bp, Artificial Sequence
  Probe for L1 gene of HPV90
cacacaaacaccctctgacacatacaaggc

SEQ ID NO: 251 - DNA, 30 bp, Artificial Sequence
  Probe for L1 gene of HPV90
acacaaacaccctctgacacatacaaggct

SEQ ID NO: 252 - DNA, 30 bp, Artificial Sequence
  Probe for L1 gene of HPV90
cacaaacaccctctgacacatacaaggctt

SEQ ID NO: 253 - DNA, 30 bp, Artificial Sequence
  Probe for L1 gene of HPV90
acaaacaccctctgacacatacaaggcttc

SEQ ID NO: 254 - DNA, 30 bp, Artificial Sequence
  Probe for L1 gene of HPV90
caaacaccctctgacacatacaaggcttcc

SEQ ID NO: 255 - DNA, 30 bp, Artificial Sequence
  Probe for L1 gene of HPV90
aaacaccctctgacacatacaaggcttcca

SEQ ID NO: 256 - DNA, 30 bp, Artificial Sequence
  Probe for L1 gene of HPV90
aacaccctctgacacatacaaggcttccaa

SEQ ID NO: 257 - DNA, 30 bp, Artificial Sequence
  Probe for L1 gene of HPV90
acaccctctgacacatacaaggcttccaat

SEQ ID NO: 258 - DNA, 30 bp, Artificial Sequence
  Probe for L1 gene of HPV91
cttgtgtgcatccactgagtctgtgctacc

SEQ ID NO: 259 - DNA, 30 bp, Artificial Sequence
  Probe for L1 gene of HPV91
ttgtgtgcatccactgagtctgtgctacct

SEQ ID NO: 260 - DNA, 30 bp, Artificial Sequence
  Probe for L1 gene of HPV91
tgtgtgcatccactgagtctgtgctaccta

SEQ ID NO: 261 - DNA, 30 bp, Artificial Sequence
  Probe for L1 gene of HPV91
gtgtgcatccactgagtctgtgctacctac

SEQ ID NO: 262 - DNA, 30 bp, Artificial Sequence
  Probe for L1 gene of HPV91
tgtgcatccactgagtctgtgctacctact

SEQ ID NO: 263 - DNA, 30 bp, Artificial Sequence
  Probe for L1 gene of HPV91
gtgcatccactgagtctgtgctacctacta

SEQ ID NO: 264 - DNA, 30 bp, Artificial Sequence
  Probe for L1 gene of HPV91
tgcatccactgagtctgtgctacctactac

SEQ ID NO: 265 - DNA, 30 bp, Artificial Sequence
  Probe for L1 gene of HPV91
ctgtgctacctactacatatgacaacacaa

SEQ ID NO: 266 - DNA, 30 bp, Artificial Sequence
  Probe for L1 gene of HPV91
tgtgctacctactacatatgacaacacaaa

SEQ ID NO: 267 - DNA, 30 bp, Artificial Sequence
  Probe for L1 gene of HPV73
tacacaggctagtagctctactacaacgta

SEQ ID NO: 268 - DNA, 30 bp, Artificial Sequence
  Probe for L1 gene of HPV73
acacaggctagtagctctactacaacgtat

SEQ ID NO: 269 - DNA, 30 bp, Artificial Sequence
  Probe for L1 gene of HPV73
cacaggctagtagctctactacaacgtatg

SEQ ID NO: 270 - DNA, 30 bp, Artificial Sequence
  Probe for L1 gene of HPV73
acaggctagtagctctactacaacgtatgc

SEQ ID NO: 271 - DNA, 30 bp, Artificial Sequence
  Probe for L1 gene of HPV73
caggctagtagctctactacaacgtatgcc

SEQ ID NO: 272 - DNA, 30 bp, Artificial Sequence
  Probe for L1 gene of HPV73
aggctagtagctctactacaacgtatgcca

SEQ ID NO: 273 - DNA, 30 bp, Artificial Sequence
  Probe for L1 gene of HPV73
ggctagtagctctactacaacgtatgccaa

SEQ ID NO: 274 - DNA, 30 bp, Artificial Sequence
  Probe for L1 gene of HPV55
tgctacaactcagtctccatctacaacata

SEQ ID NO: 275 - DNA, 30 bp, Artificial Sequence
  Probe for L1 gene of HPV55
gctacaactcagtctccatctacaacatat

SEQ ID NO: 276 - DNA, 30 bp, Artificial Sequence
  Probe for L1 gene of HPV55
ctacaactcagtctccatctacaacatata

SEQ ID NO: 277 - DNA, 30 bp, Artificial Sequence
  Probe for L1 gene of HPV55
acaactcagtctccatctacaacatataat

SEQ ID NO: 278 - DNA, 30 bp, Artificial Sequence
  Probe for L1 gene of HPV55
actcagtctccatctacaacatataatagt

SEQ ID NO: 279 - DNA, 30 bp, Artificial Sequence
  Probe for L1 gene of HPV57
tttgtgtgccactgtaaacacagaaactaa

SEQ ID NO: 280 - DNA, 30 bp, Artificial Sequence
  Probe for L1 gene of HPV57
ttgtgtgccactgtaaacacagaaactaat

SEQ ID NO: 281 - DNA, 30 bp, Artificial Sequence
  Probe for L1 gene of HPV57
tgtgtgccactgtaaacacagaaactaatt

SEQ ID NO: 282 - DNA, 30 bp, Artificial Sequence
  Probe for L1 gene of HPV57
gtgtgccactgtaaacacagaaactaatta

SEQ ID NO: 283 - DNA, 30 bp, Artificial Sequence
  Probe for L1 gene of HPV57
tgtgccactgtaaacacagaaactaattat

SEQ ID NO: 284 - DNA, 30 bp, Artificial Sequence
  Probe for L1 gene of HPV57
gtgccactgtaaacacagaaactaattata

SEQ ID NO: 285 - DNA, 30 bp, Artificial Sequence
  Probe for L1 gene of HPV57
actgtaaacacagaaactaattataaggcc

SEQ ID NO: 286 - DNA, 30 bp, Artificial Sequence
  Probe for L1 gene of HPV57
ctgtaaacacagaaactaattataaggcct

(19) United States
(12) Patent Application Publication
(10) Pub. No.: US 2009/0088344 A1
(43) Pub. Date: Apr. 2, 2009

(54) Method Selecting Highly Specific Probes For HPV Genotype Analysis and the Probes Thereof

(75) Inventors: Yong-Ku Cho, Seoul (KR); Chang-Il Hwang, Gyounggi-do (KR)

Correspondence Address: LEXYOUME IP GROUP, LLC, 5180 PARKSTONE DRIVE, SUITE 175, CHANTILLY, VA, 20151, US

(73) Assignee: BIOMEDLAD, CO., Ansan-si (KR)

(21) Appl. No.: 11/917,263

(22) PCT Filed: Jul. 5, 2005
(86) PCT No.: PCT/KR2005/002125

§ 371 Date: Dec. 12, 2007

(30) Foreign Application Priority Data
Jul. 5, 2004 (KR) 10-2004-0052068

(51) Int. Cl. C40B 40/06; G01N 33/50; C07H 21/00

(52) U.S. Cl. 506/16; 436/94; 536/24.32

(57) ABSTRACT

A method for selecting a highly specific probe among a predetermined range of nucleotide sequences comprises:
setting a group of nucleotide sequences among the predetermined range of nucleotide sequences; setting a range of nucleotide sequences in the group of nucleotide sequences; selecting first candidate probes having a certain length within the range of nucleotide sequences; selecting second candidate probes whose melting temperature with target nucleic acids for the first candidate probes is in an appropriate range; selecting third candidate probes whose melting temperature with a specific set of nucleotide sequences is lower than a hybridization temperature; and selecting fourth candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

### TECHNICAL FIELD

The present invention relates to a method for selecting a highly specific probe from a predetermined range of nucleotide sequences and a highly specific probe selected by using the same; and more particularly, to a method for selecting a highly specific probe including nucleic acids for a human papillomavirus (HPV) genotype analysis and a highly specific probe selected by using the same.

### BACKGROUND ART

Nucleic acids are high molecular organic substances. Deoxyribonucleic acid (DNA) and ribonucleic acid (RNA) existing within living cells are representative examples of such nucleic acids. Although not discovered in nature, artificially synthesized nucleic acids such as peptide nucleic acid (PNA), locked nucleic acid (LNA) and so forth can be useful as well.

Various methods of detecting a nucleic acid from a certain specimen have been introduced. For instance, a liquid hybridization method, a southern blot method, a dot blot method, an in situ hybridization method, a microtiter plate hybridization method, and a line probe assay method are commonly known detection methods.

Recently, a method of detecting various sequences of DNA and RNA included in one specimen using a DNA chip in single experiment is introduced and used widely. Especially, this recently introduced detection method makes it possible to analyze a large quantity of sequences with high sensitivity and thus, this detection method will become a useful tool for various research studies on nucleic acids and will be more widely implemented for genetic disease diagnoses.

A probe including nucleic acids is generally an essential element for detecting the aforementioned nucleic acids. Specifically, a nucleic acid probe having high specificity is important to detect nucleic acids with various sequences within a specimen. Also, the nucleic acid probes should have high sensitivity to detect even a tiny amount of a nucleic acid included in a specimen. In the case of using a number of probes, sensitivity between the probes should be maintained consistent.

Several procedures are performed to select such a nucleic acid probe; they are, (1) analysis of a unique nucleotide sequence that belongs to a target nucleic acid to be detected, (2) analysis of a melting temperature (Tm) of the nucleic acid probe and the target nucleic acid, and (3) analysis of a secondary structure of the nucleic acid probe.

In a conventional detection method, a nucleotide sequence of an intended target nucleic acid is compared with that of another nucleic acid by aligning these nucleotide sequences or, a similar nucleotide sequence is analyzed through a BLAST search using an internet database. The secondary structure of the nucleic acid is generally important for sensitivity of the nucleic acid probe. Typically, the conventional method analyzes secondary structures of nucleic acids and selects a probe including a minimum number of secondary structures. The reason for selecting the probe with the minimum number of secondary structures is because the secondary structure of the probe tends to decrease the sensitivity. However, in the case that the specificity of nucleic acid probe is a crucial factor, for instance, when several tens or hundreds to several thousands of probes such as a DNA chip are used to detect a specific nucleic acid within a specimen, using the secondary structure of nucleic acid is a generally known method to improve the specificity. In U.S. Pat. No. 5,780,610 issued to M. L. Collins et al. on Jul. 14, 1998, entitled “Reduction of Nonspecific Hybridization by Using Novel Base-Paring Schemes,” specificity of a probe is improved by artificially synthesizing a secondary structure using a non-natural nucleotidic unit. Also, U.S. Pat. No. 6,114,121 issued to J. Fujiwara et al. on Sep. 5, 2000, entitled “Nucleic Acid Probe Molecule of Hairpin-Shape Structure and Method for Detecting Nucleic Acid Using the Same” teaches a method for detecting a target nucleic acid in a double helical structure through generating a complex between a probe with a secondary structure and RecA. Especially, in U.S. Pat. No. 6,596,490 issued to N. Dattagupta on Jul. 22, 2003, entitled “Nucleic Acid Hairpin Probes and Uses Thereof,” a method of detecting a nucleic acid within a specimen using a probe with a hairpin structure is suggested.

Despite the above suggested conventional detection methods, it is necessary to have a probe which is more effective in detecting a genotype of HPV. HPV is one main cause of cervical intraepithelial neoplasia, which is a pre-stage of cervical cancer. Currently, more than 70 types of HPV have been reported in an article by E. O. Wiley, Phylogentics, John Wiley and Sons, New York, 1981. Also, it is learned in an article by C. Clavel et al., British Journal of Cancer, Vol. 84, pp. 1616-1623, 2001 that HPV infection of a specific genotype is highly associated with neoplasia. Therefore, it is recently considered important to develop an effective method of detecting individual nucleic acids of HPV according to different genotypes. For instance, a genotype detection kit using a DNA chip is disclosed in Korean application No. 10-2000-0013161 (Korean patent No. 0382703) issued to S. K. Kim on Apr. 21, 2003, entitled “Diagnosis Kit for Genotyping of Human Papillomavirus and manufacturing method for thereof.”

Although a probe for detecting a genotype of HPV should have a high level of specificity to distinguish various genotypes of HPV, the conventional detection methods may still have limitations to select a highly specific probe effectively.

### DISCLOSURE OF INVENTION

### Technical Problem

It is, therefore, an object of the present invention to provide a method for selecting a highly specific probe including nucleic acids from a predetermined range of nucleotide sequences and a highly specific probe selected by using the same.

It is another object of the present invention to provide a method for selecting a highly specific probe including nucleic acids to analyze a genotype of HPV and a highly specific probe selected by using the same.

It is a further another object of the present invention to provide a probe that can anneal with DNA and RNA of HPV and has a stable secondary structure in hybridization reaction conditions.

In accordance with one aspect of the present invention, there is provided a method for selecting a highly specific probe among a predetermined range of nucleotide sequences, including: setting a group of nucleotide sequences to be analyzed among the predetermined range of nucleotide sequences; setting a range of nucleotide sequences of probes to be selected in the group of nucleotide sequences; selecting first candidate probes whose length ranges from approximately 20 mer to approximately 50 mer within the range of nucleotide sequences of the probes; selecting second candidate probes whose melting temperature with target nucleic acids for the first candidate probes ranges from approximately 50° C. to approximately 80° C. among the first candidate probes; selecting third candidate probes whose melting temperature with nucleotide sequences except for the nucleotide sequences of the target nucleic acids among the group of nucleotide sequences is lower than a hybridization temperature among the second candidate probes; and selecting fourth candidate probes among the third candidate probes, wherein a secondary structure of each fourth candidate probe has a melting temperature lower than the hybridization temperature by approximately 5° C. to approximately 10° C. and higher than lower than a temperature that is lower than the hybridization temperature by approximately 5° C. to approximately 10° C.

The first candidate probes selected within the range of nucleotide sequences of probes to be selected may have a preferable length ranging from approximately 30 mer to approximately 35 mer. The melting temperature of the target nucleic acids for the first candidate probes and the second probe candidates may range preferably from approximately 65° C. to approximately 75° C.

The melting temperature of the third candidate probes and the nucleotide sequences except for the nucleotide sequences of the target nucleic acids may be lower than the hybridization temperature by approximately 5° C. to approximately 10° C. and lower than a temperature that may be lower than the hybridization temperature by approximately 5° C. to approximately 10° C. Also, the melting temperature of the secondary structure of each fourth candidate probe may be higher than the hybridization temperature.

In accordance with another aspect of the present invention, there is provided a method for selecting a highly specific probe for a HPV genotype analysis, including: selecting at least one nucleotide sequences selected from the group consisting of nucleotide sequences of an L1 gene, an E6 gene and an E1 gene according to each HPV genotype; selecting first candidate probes whose length ranges from approximately 20 mer to approximately 50 mer within the nucleotide sequences; selecting second candidate probes whose melting temperature with target nucleic acids for the first candidate probes ranges from approximately 50° C. to approximately 80° C. among the first candidate probes; selecting third candidate probes whose melting temperature with nucleotide sequences except for the nucleotide sequences of the target nucleic acids among the nucleotide sequences is lower than a hybridization temperature among the second candidate probes; and selecting fourth candidate probes among the third candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

In each of the HPV genotypes, the nucleotide sequences of the L1 gene may be preferably selected. Preferably, the first candidate probes selected within the nucleotide sequences may have a length ranging from approximately 30 mer to approximately 35 mer.

The melting temperature of the target nucleic acids for the first candidate probes and the second probe candidates may range from approximately 65° C. to approximately 75° C. Also, the melting temperature of the third candidate probes and the nucleotide sequences except for the nucleotide sequences of the target nucleic acids may be lower than the hybridization temperature by approximately 5° C. to approximately 10° C. and lower than a temperature that may be lower than the hybridization temperature by approximately 5° C. to approximately 10° C. Also, the melting temperature of the secondary structure of each fourth candidate probe may be higher than the hybridization temperature.

In accordance with still another aspect of the present invention, there is provided a method for selecting a highly specific probe for a HPV genotype analysis, wherein at least one pair is selected from a pair of SEQ ID NOS: 301 and 302 or a pair of SEQ ID NOS: 303 and 304 which are selected from the group consisting of primers for HPV and, a portion of a gene that is pertained to each HPV genotype and amplified by the selected primer pair is determined, the method including: selecting first candidate probes whose length ranges from approximately 20 mer to approximately 50 mer within the portion of the gene; selecting second candidate probes whose melting temperature with target nucleic acids for the first candidate probes ranges from approximately 50° C. to approximately 80° C. among the first candidate probes; selecting third candidate probes whose melting temperature with nucleotide sequences except for nucleotide sequences of the target nucleic acids among a group of nucleotide sequences to be analyzed is lower than a hybridization temperature among the second candidate probes; and selecting fourth candidate probes among the third candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

The first candidate probes selected within the selected group of nucleotide sequences to be analyzed may have a length ranging from approximately 30 mer to approximately 35 mer. The melting temperature of the target nucleic acids for the first candidate probes and the second probe candidates may range preferably from approximately 65° C. to approximately 75° C.

The melting temperature of the third candidate probes and the nucleotide sequences except for the nucleotide sequences of the target nucleic acids may be lower than the hybridization temperature by approximately 5° C. to approximately 10° C. and lower than a temperature that may be lower than the hybridization temperature by approximately 5° C. to approximately 10° C. Also, the melting temperature of the secondary structure of each fourth candidate probe may be higher than the hybridization temperature.

In accordance with a further another aspect of the present invention, there is provided a probe that is complementary with DNA and RNA of HPV, wherein the probe is selected from the group consisting of oligonucleotides having nucleotide sequences of SEQ ID NOS: 1 to 286 by employing one method as described above.

Among the SEQ ID NOS: 1 to 286, the probe may be selected from the group consisting of oligonucleotides having nucleotide sequences of SEQ ID NOS: 1, 7, 14, 26, 27, 34, 41, 50, 54, 59, 61, 66, 75, 80, 83, 89, 97, 109, 113, 125, 140, 151, 166, 172, 184, 189, 207, 213, 217, 228, 236, 249, 264, 270, 276, and 283. Preferably, the probe may be selected from the group consisting of oligonucleotides having nucleotide sequences of SEQ ID NOS: 1, 7, 26, 27, 34, 41, 50, 54, 59, 61, 66, 75, 80, 83, 89, 97, 109, 113, 125, and 140.

In accordance with a further another aspect of the present invention, there is provided a DNA chip for HPV including at least one probes selected from the group consisting of the aforementioned probes.

### BRIEF DESCRIPTION OF THE DRAWINGS

Other objects and aspects of the invention will become apparent from the following description of the embodiments with reference to the accompanying drawings, in which:

FIG. 1 is a mimetic diagram showing various exemplary secondary structures of nucleic acids defined by an embodiment of the present invention;

FIG. 2 is a mimetic diagram showing a DNA chip including conventional probes presented with different genotypes and positions for the comparison and evaluation purpose with respect to probes according to an embodiment of the present invention;

FIG. 3 is a mimetic diagram showing a DNA chip including probes presented with different genotypes and positions according to an embodiment of the present invention;

FIG. 4 is a mimetic diagram showing an analysis result of DNA of HPV 26 using the DNA chip shown in FIG. 2; and

FIG. 5 is a mimetic diagram showing an analysis result of DNA of HPV 26 using the DNA chip shown in FIG. 3.

### BEST MODE FOR CARRYING OUT THE INVENTION

Hereinafter, detailed description of the present invention will be provided with reference to accompanying drawings.

The present invention is focused on a method for selecting a highly specific probe including nucleic acids from a predetermined range of nucleotide sequences. According to the present invention, nucleic acids include naturally discovered nucleic acids in living cells such as DNA and RNA as well as non-natural nucleic acids such as PNA and LNA. In the case that a length of DNA is less than approximately 100 mer, this short DNA is called oligonucleotide. Also, the probe according to the present invention can be defined as a molecule of nucleic acid combining strongly with a nucleic acid having one predetermined nucleotide sequence to be detected in a specimen. The predetermined nucleotide sequence is called a target nucleic acid for the probe. Also, the strong annealing takes place when the nucleotide sequence of the probe and the nucleotide sequence of the target nucleic acid are completely complementary with each other. Also, a candidate probe is a molecule of nucleic acid that can be selected as a probe. The term ‘specificity’ according to the present invention indicates a degree of annealing that one probe anneals only with a nucleic acid having a predetermined nucleotide sequence but not with other nucleic acids having nucleotide sequences different from the nucleotide of the annealed nucleic acid. That is, a probe with a high level of specificity is less likely to anneal with a nucleic acid having a nucleotide sequence that is not a predetermined nucleotide sequence of a target nucleic acid.

A specimen defined in the present invention is a liquid or solid substance taken from a clinically meaningful body or a combination thereof and, includes a tissue, a cell scrapped from a tissue, a fixated cell, and various types of body fluids such as blood.

According to the present invention, a hybridization reaction or hybridization is a procedure that a target nucleic acid within a specimen or an amplification product of a target nucleic acid anneals with a probe by making the probe to contact a certain specimen or a substance extracted from a specimen. Also, hybridization temperature is a temperature at which the hybridization reaction occurs. Hybridization conditions include hybridization temperature and ionic concentration of a solution.

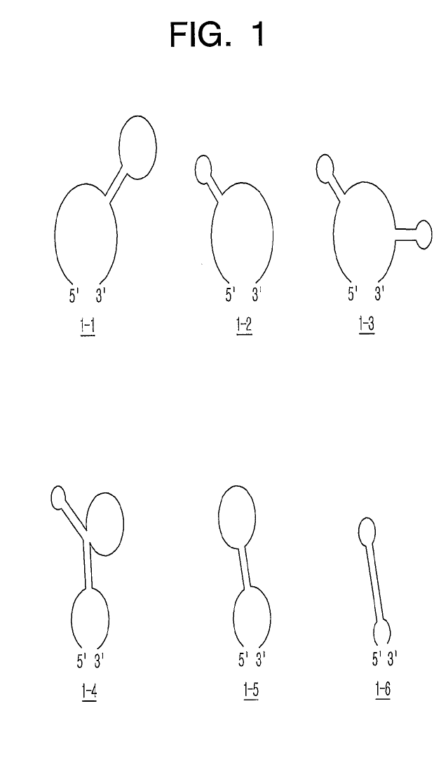

A secondary structure of a nucleic acid in the present invention is a molecular type that a single-stranded nucleic acid molecule forms a partial double helix as a portion of the nucleic acid anneals with another portion of the nucleic acid through a self-structural transformation without changing a nucleotide sequence and a further another portion of the nucleic acid exists in a single strand. The secondary structure of the nucleic acid according to the present invention includes various types of molecular structures as shown in FIG. 1 but is not limited by the embodied molecular structures.

A suggested method for selecting a highly specific probe in accordance with the present invention includes procedures proceeding with a computer simulation as follows: they are, (1) setting of a group of nucleotide sequences to be analyzed; (2) setting of a range of nucleotide sequences for selecting probes among the selected group of nucleotide sequences; (3) selection of first candidate probes each having a predetermined length within the range of nucleotide sequences; (4) selection of second candidate probes among the first candidate probes, wherein the second candidate probes have an appropriate range of a melting temperature with respective target nucleic acids for the first candidate probes; (5) selection of third candidate probes that have a melting temperature with nucleotide sequences from the above nucleotide sequence group except for the nucleotide sequences of the target nucleic acids lower than the appropriate range of melting temperature; and (6) selection of fourth candidate probes, wherein a melting temperature of a secondary structure of the individual fourth candidate probe is greater than the appropriate range of melting temperature.

In accordance with the present invention, the group of nucleotide sequences to be analyzed at the procedure (1) is a collection of nucleotide sequences including a gene to be analyzed or a portion of other nucleotide sequences and complementary nucleotide sequences thereof. Among the group of nucleotide sequences to be analyzed, a range of the nucleotide sequences to be detected by probes is selected and then, candidate probes are selected from the range of the nucleotide sequences. One example of the candidate probe selection method will descried hereinafter.

In the case of detecting a genotype of HPV, a L1 gene can be selected as a range of nucleotide sequences to be detected in each of HPV genotypes. Then, candidate probes are selected from the nucleotide sequences of the L1 gene amplified by primers of SEQ ID NOS: 301 and 302.

In more detail of the above described probe selection method, if the nucleotide sequence of the L1 gene amplified via polymerase chain reaction (PCR) is set to have a length of approximately 100 mer while the probe is set to have a length of approximately 30 mer, the nucleotide sequence of the L1 gene is cut off for every 30 mer from a first nucleotide, thereby providing first candidate probes. Then, the nucleotide sequence of the L1 gene is cut off for every 30 mer from a second nucleotide, thereby providing second candidate probes. The nucleotide sequence is cut off consecutively as described above to provide other candidate probes. Afterwards, at least one targeted probe is selected in consideration of melting temperature and other conditions.

In the procedure (3) of the highly specific probe selection method, the pre-determined length is preferably in a range of approximately 20 mer to approximately 50 mer, more preferably in a range of approximately 30 mer to approximately 35 mer.

If the predetermine length is less than approximately 20 mer, the sensitivity of the probe selection method may decrease due to a weak bonding force. In contrast, if the predetermined length is greater than approximately 50 mer, a chance of non-specific annealing such as cross-reactions with other probes may increase.

One example of calculating a melting temperature described in the procedure (4) of the highly specific probe selection method is a nearest-neighbor method introduced in an article by John SantaLucia Jr., Proc. Natl. Acad. Sci. USA, Vol. 95, pp. 1460-1465, 1998. Also, the appropriate range of melting temperature is between approximately 50° C. and approximately 80° C., more preferably, between approximately 65° C. and approximately 75° C.

The melting temperature increases as a probe has a high matching level due to a high complementary characteristic of the probe with respect to a nucleotide sequence of a target nucleic acid within a specimen. Also, as the number of base parings between Guanine (G) and Cystine (C) increases, the melting temperature increases as well. For instance, if a melting temperature of a probe of HPV 16 among various HPV genotypes and a target nucleic acid of such probe is assumed to be approximately 75° C., a melting temperature of HPV 18 and the probe of HPV 16 is lower than approximately 75° C. as the specificity of the above probe of HPV 16 increases. The range of melting temperature determined as above is between approximately 50° C. and approximately 80° C. If the melting temperature is lower than approximately 50° C., a bonding force may become weak. A typical melting temperature is less than approximately 80° C.

The appropriate temperature described in the procedure (5) of the highly specific probe selection method is preferably a temperature of a hybridization condition. More preferably, the appropriate temperature is lower than the temperature of the hybridization condition by approximately 5° C. to approximately 10° C. In other words, the appropriate temperature is lower than a melting temperature of a probe and a target nucleic acid as the specificity of the probe increases. Thus, to maintain a desired level of specificity, the appropriate temperature is preferably a temperature of the hybridization condition that is lower than approximately 50° C., which is in a range of a minimum melting temperature set at the procedure (4). Typically, the temperature of the hybridization condition is approximately 40° C.

In an article by M. Zuker, Nucleic Acids Res. Vol. 31 (13), pp. 3406-3415, 2003, one example of calculating a melting temperature of a secondary structure described in the procedure (6) is described. Also, the appropriate temperature mentioned in the procedure (6) is preferably lower than the temperature of the hybridization condition by approximately 5° C. to approximately 10° C. More preferably, the appropriate temperature described in the procedure (6) is the temperature of the hybridization condition. A probe with a stable secondary structure has a decreased level of sensitivity, and a melting temperature of such probe and a target nucleic acid or of such probe and nucleotide sequences except for the target nucleic acid decreases. The specificity of the probe can be maintained when the melting temperature of the probe and the target nucleic acid is higher than the melting temperature of the probe and other nucleotide sequences except for the target nucleic acid. Thus, the specificity of the probe can be maintained when the melting temperature in the procedure (6) is higher than the appropriate temperature in the procedure (5). Hence, the melting temperature in the sixth procedure (6) is preferably higher than a temperature that is lower than the temperature of the hybridization condition by approximately 5° C. to approximately 10° C.

The probe selection method according to the present invention can be applicable when a group of nucleotide sequences for selecting one or more probe is predetermined. Examples of such nucleotide sequence group are a L1 gene, an E1 gene and an E6 gene pertained to each genotype of HPV. A gene portion per HPV genotype amplified via individual primers is another example of such nucleotide sequence group. The table 1 provided below shows the above primers amplifying the gene portion.

TABLE 1

| SEQ | | | |
|---|---|---|---|
| ID | Name of | | Number of |
| No: | Primer | Sequence (5′-3′) | nucleotides |
| 301 | Gp5d+ | TTTKTTACHGTKGTDGATACYAC | 23 |
| 302 | Gp6d+ | GAAAHATAAAYTGYAADTCATAYTC | 25 |
| 303 | Gp5d2 | TTTKTWACHGTKGTDGAYACHWC | 23 |
| 304 | Gp6d2 | GAAAHAYAAAYTGYAADTCAWAYTC | 25 |

Herein, according to the international nomenclature for degenerated bases, those symbols R, Y, M, K, S, W, V, H, B, and D indicate (A or G), (C or T), (A or C), (G or T), (G or C), (A or T), (A or C or G), (A or C or T), (G or T or C), and (A or G or T), respectively and, these symbols are well known to those ordinary people skilled in the art.

A suggested method for effectively selecting a highly specific probe to analyze HPV genotypes includes: (1) selecting first candidate probes having a predetermined length from a group of nucleotide sequences selected from the group consisting of a L1 gene, an E6 gene and an E1 gene according to each genotype of HPV; (2) selecting second candidate probes among the first candidate probes, wherein the second candidate probes have a melting temperature with target nucleic acids for the first candidate probes in an appropriate range; (3) selecting third candidate probes among the second candidate probes, wherein the third candidate probes have a melting temperature with nucleotide sequences from the above nucleotide sequence group except for the nucleotide sequences of the target nucleic acids lower than the appropriate range of melting temperature; and (4) selecting fourth candidate probes among the third candidate probes, wherein a melting temperature of a secondary structure of the individual fourth candidate probe is greater than the appropriate range of melting temperature.

In a portion of a gene according to individual genotypes of HPV amplified by at least one pairs of primers selected from a pair of SEQ ID NOS: 301 and 302, or a pair of SEQ ID NOS: 303 and 304 selected the group consisting of those primers for HPV, a method for selecting a probe for a HPV genotype analysis includes: (1) selecting first candidate probes each with a predetermined length; (2) selecting second candidate probes among the first candidate probes, wherein the second candidate probes have a melting temperature with target nucleic acids for the first candidate probes in an appropriate range; (3) selecting third candidate probes among the second candidate probes, wherein the third candidate probes have a melting temperature with nucleotide sequences from the above nucleotide sequence group except for the nucleotide sequences of the target nucleic acids lower than the appropriate range of melting temperature; and (4) selecting fourth candidate probes among the third candidate probes, wherein a melting temperature of a secondary structure of the individual fourth candidate probe is higher than the appropriate range of melting temperature.

In accordance with the present invention, by using the above described probe selection methods, it is possible to measure melting temperatures of probes set forth in SEQ ID NOS: 1 to 286 and target nucleic acids thereof and a melting temperature of a secondary structure of each probe. Also, a probe that forms a stable secondary structure under hybridization reaction conditions is selected.

According to the present invention, probes that are complementary with DNA or RNA of HPV are selected from the group consisting of oligonucleotides set forth in SEQ ID NOS: 1 to 286 through employing one of the above descried selection methods.

Among those probes selected by one of the embodied selection methods, those probes, which are complementarily paired with DNA or RNA of HPV, are selected from the group consisting of oligonucleotides set forth in SEQ ID NOS: 1, 7, 14, 26, 27, 34, 41, 50, 54, 59, 61, 66, 75, 80, 83, 89, 97, 109, 113, 125, 140, 151, 166, 172, 184, 189, 207, 213, 217, 228, 236, 249, 264, 270, 276, and 283. It is more preferable to select those probes complementarily paired with DNA or RNA of HPV from the group consisting of oligonucleotides set forth in SEQ ID NOS: 1, 7, 26, 27, 34, 41, 50, 54, 59, 61, 66, 75, 80, 83, 89, 97, 109, 113, 125, and 140.

Hereinafter, embodiments in accordance with the present invention will be described in detail. It should be appreciated that the embodiments are provided for the purpose that one ordinary skilled in the art would be able to understand the present invention, and modifications in various manners and the scope of the present invention are not limited by the embodiments described herein.

### EXAMPLE 1

### Selection of Probe for Genotype of HPV 67

The selection of one or more probes for a genotype of HPV 67 includes: (1) setting a group of nucleotide sequences to be analyzed; (2) setting a range of nucleotide sequences used for selecting a specific probe; (3) selecting first candidate probes each with a predetermined length; (4) calculating a melting temperature of the individual first candidate probe and a target nucleic acid thereof; (5) calculating a melting temperature of the individual second candidate probe selected from the above procedure (4) and nucleotide sequences from the nucleotide sequence group except for the nucleotide sequences of the target nucleic acids; and (6) calculating a melting temperature of a secondary structure of the individual third candidate probe selected from the above procedure (5).

In more detail of the specifically embodied probe selection method, a group of nucleotide sequences to be analyzed includes nucleotide sequences of a L1 gene of HPV genotypes including 16, 18, 31, 33, 35, 39, 45, 51, 52, 56, 58, 59, 66, 68, 69, 6, 11, 34, 40, 42, 43, 44, 26, 30, 54, 70, 72, 82, 53, 61, 62, 67, 71, 74, 83, 84, 85, 89, 90, 91, CP8304, 73, MM4, MM7, MM8, MM9, CP6108, ISO39, 55, and 57 and complementary nucleotide sequences thereof. Among the selected group of nucleotide sequences, a range of nucleotide sequences of HPV 67 that can be amplified by primers of SEQ ID NOS: 301 and 302 or SEQ ID NOS: 303 and 304 is selected. Within the determined range of nucleotide sequences of HPV 67, approximately 110 candidate probes whose length is approximately 30 mer are selected. Among the selected 110 candidate probes, those probes whose melting temperature with target nucleic acids is in a range of approximately 65° C. to approximately 75° C. under hybridization conditions are selected. Particularly, the melting temperature is calculated by using the nearest-neighbor method introduced by SantaLucia. Then, among the above selected candidate probes whose melting temperature is in a range of approximately 65° C. to approximately 75° C., those probes whose melting temperature with nucleotide sequences from the above selected nucleotide sequence group except for the nucleotide sequences of the target nucleic acids is less than approximately 35° C. under hybridization conditions. As shown in Table 2 below, approximately 21 candidate probes are selected.

TABLE 2

| | | | Tm with |
|---|---|---|---|
| | | Tm with | Other |
| | | Target | nucleotide |
| Name | | Nucleic | Sequences |
| of | | Acid | (Type, |
| Probe | Base Sequence (5′-3′) | (° C.) | Tm (° C.)) |
| 67_41 | TTTATGTTCTGAGGAAAAATCAGA | 65.8 | 52 (19.8) |
| | GGCTAC | | |
| 67_42 | TTATGTTCTGAGGAAAAATCAGAG | 67.5 | 52 (19.6) |
| | GCTACA | | |
| 67_44 | ATGTTCTGAGGAAAAATCAGAGGC | 66.2 | 52 (20.1) |
| | TACATA | | |
| 67_45 | TGTTCTGAGGAAAAATCAGAGGCT | 66.8 | 52 (15.7) |
| | ACATAC | | |
| 67_46 | GTTCTGAGGAAAAATCAGAGGCTA | 67.8 | 52 (13.7) |
| | CATACA | | |
| 67_47 | TTCTGAGGAAAAATCAGAGGCTAC | 67.5 | 52 (7.8) |
| | ATACAA | | 16 (6.9) |
| 67_48 | TCTGAGGAAAAATCAGAGGCTACA | 67.3 | 16 (13.9) |
| | TACAAA | | |
| 67_49 | CTGAGGAAAAATCAGAGGCTACAT | 67.6 | 16 (19.6) |
| | ACAAAA | | |
| 67_50 | TGAGGAAAAATCAGAGGCTACATA | 65.9 | 16 (26.0) |
| | CAAAAA | | |
| 67_51 | GAGGAAAAATCAGAGGCTACATAC | 66.4 | 16 (30.3) |
| | AAAAAT | | |
| 67_52 | AGGAAAAATCAGAGGCTACATACA | 66.7 | 16 (27.6) |
| | AAAATG | | |
| 67_53 | GGAAAAATCAGAGGCTACATACAA | 65.6 | 16 (27.6) |
| | AAATGA | | |
| 67_54 | GAAAAATCAGAGGCTACATACAAA | 64.4 | 16 (27.6) |
| | AATGAA | | |
| 67_55 | AAAAATCAGAGGCTACATACAAAA | 65.1 | 16 (27.6) |
| | ATGAAA | | |
| 67_56 | AAAATCAGAGGCTACATACAAAAA | 63.5 | 16 (21.0) |
| | TGAAAA | | |
| 67_57 | AAATCAGAGGCTACATACAAAAAT | 65.6 | 16 (28.1) |
| | GAAAAC | | |
| 67_58 | AATCAGAGGCTACATACAAAAATG | 66.8 | 16 (30.8) |
| | AAAACT | | |
| 67_59 | ATCAGAGGCTACATACAAAAATGA | 66.8 | 16 (33.4) |
| | AAACTT | | |
| 67_60 | TCAGAGGCTACATACAAAAATGAA | — | 16 (35.0) |
| | AACTTT | | |
| 67_61 | CAGAGGCTACATACAAAAATGAAA | 65.1 | 16 (34.0) |
| | ACTTTA | | |

Afterwards, a melting temperature of a secondary structure of each selected candidate probe, i.e., each of the 21 candidate probes, is calculated based on a method introduced by Zuker. Among the 21 candidate probes, approximately 10 candidate probes whose melting temperature is greater than approximately 40° C. under hybridization conditions are selected. The selected 10 candidate probes are shown in Table 3 below.

TABLE 3

| | | Tm with | |
|---|---|---|---|
| | | Target | Tm of |
| Name | | Nucleic | Secondary |
| of | | Acid | Structure |
| Probe | Base Sequence (5′□3′) | (° C.) | (° C.) |
| 67_41 | TTTATGTTCTGAGGAAAAATCAGA | 65.8 | 54.8 |
| | GGCTAC | | |
| 67_42 | TTATGTTCTGAGGAAAAATCAGAG | 67.5 | 54.8 |
| | GCTACA | | |
| 67_44 | ATGTTCTGAGGAAAAATCAGAGGC | 66.2 | 52.1 |
| | TACATA | | |
| 67_45 | TGTTCTGAGGAAAAATCAGAGGCT | 66.8 | 54.8 |
| | ACATAC | | |
| 67_46 | GTTCTGAGGAAAAATCAGAGGCTA | 67.8 | 54.8 |
| | CATACA | | |
| 67_47 | TTCTGAGGAAAAATCAGAGGCTAC | 67.5 | 54.8 |
| | ATACAA | | |
| 67_48 | TCTGAGGAAAAATCAGAGGCTACA | 67.3 | 54.8 |
| | TACAAA | | |
| 67_49 | CTGAGGAAAAATCAGAGGCTACAT | 67.6 | 51.1 |
| | ACAAAA | | |
| 67_53 | GGAAAAATCAGAGGCTACATACAA | 65.6 | 47.2 |
| | AAATGA | | |

Those probes selected based on the above selection procedures are determined as a probe for HPV 67.

### EXPERIMENTAL EXAMPLE 1

### Specificity Comparison Between Selected Probes According To Embodied Method of the Present Invention

Hereinafter, detailed description of an experiment for the specificity comparison will be provided.

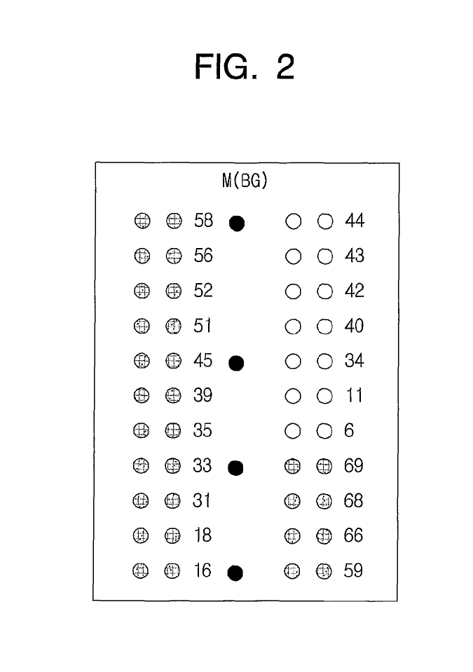

A DNA chip shown in FIG. 2 having different types of probes selected from provided nucleotide sequences set forth in SEQ ID NOS: 305 through 326 revealed in Korean Application No. 2003-0027178 (Korean Patent No. 0452163) issued to S. W. Yoon on Sep. 30, 2004, entitled “Genotyping Kit for Diagnosis of Human Papillomavirus Infection” is fabricated. Table 4 provided below shows the nucleotide sequences of SEQ ID NOS: 305 through 326.

TABLE 4

| SEQ | | |
|---|---|---|
| ID | HPV | |
| No: | Genotype | Base Sequence (5′-3′) |
| 305 | 16 | TATGTGCTGCCATATCTACTTCAGAAACTACATA |
| 306 | 18 | TGCTTCTACACAGTCTCCTGTACCTGGGCA |
| 307 | 31 | TGTTTGTGCTGCAATTGCAAACAGTGATAC |
| 308 | 33 | TTTATGCACACAAGTAACTAGTGACAGTAC |
| 309 | 35 | TTCTGCTGTGTCTTCTAGTGACAGTACATA |
| 310 | 39 | TCTACCTCTATAGAGTCTTCCATACCTTCT |
| 311 | 45 | ACACAAAATCCTGTGCCAAGTACATATGAC |
| 312 | 51 | AGCACTGCCACTGCTGCGGTTTCCCCAACA |
| 313 | 52 | TGCTGAGGTTAAAAAGGAAAGCACATATAA |
| 314 | 56 | TATTAGTACTGCTACAGAACAGTTAAGTAA |
| 315 | 58 | CACTGAAGTAACTAAGGAAGGTACATATAA |
| 316 | 59 | TCTACTACTTCTTCTATTCCTAATGTATAC |
| 317 | 66 | CTAAAAGCACATTAACTAAATATGATGCCC |
| 318 | 6 | ATCCGTAACTACATCTTCCACATACACCAA |
| 319 | 11 | ATCTGTGTCTAAATCTGCTACATACACTAA |
| 320 | 34 | GGTACACAATCCACAAGTACAACTGCACCA |
| 321 | 40 | CTTATGTGCTGCCACACAGTCCCCCACACC |
| 322 | 42 | CTGCAACATCTGGTGATACATATACAGCTG |
| 323 | 44 | GCCACTACACAGTCCCCTCCGTCTACATAT |
| 324 | 68 | TTTGTCTACTACTACTGAATCAGCTGTACCAAA |
| 325 | 69 | AATCTGCATCTGCCACTTTTAAACCATCAGATT |
| 326 | 43 | CCTCTACTGACCCTACTGTGCCCAGTACAT |

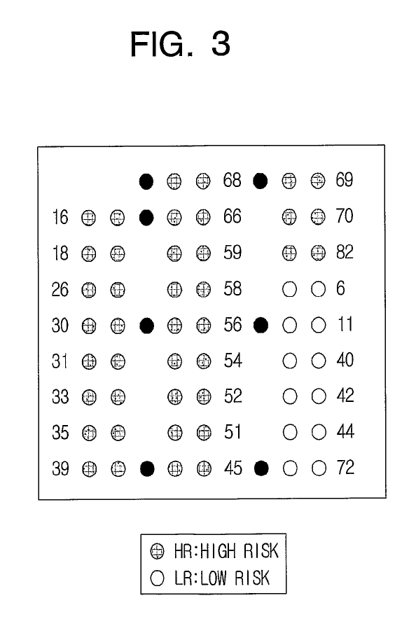

FIG. 3 shows a DNA chip having different types of probes selected from nucleotide sequences of SEQ ID NOS: 1, 7, 26, 27, 34, 41, 50, 54, 59, 61, 66, 75, 80, 83, 89, 97, 109, 113, 125, and 140.

A plasmid DNA corresponding to HPV 26 is amplified through PCR using primers of the SEQ ID NOS: 301 and 302. More specifically, the amplification of the plasmid DNA is achieved through sequential processes by first repeating a cycle 5 times, wherein the cycle includes: degenerating the plasmid DNA at approximately 94° C. for approximately 5 minutes; degenerating the plasmid DNA at approximately 94° C. for approximately 1 minute; annealing the primers at approximately 50° C. for approximately 2 minutes; and extending the primer annealed plasmid DNA at approximately 72° C. for approximately 30 seconds. After the repetition of the described cycle, another cycle including: degenerating the resulting plasmid DNA at 94° C. for approximately 1 minute; annealing the primers at approximately 50° C. for approximately 2 minutes; and extending the primer annealed plasmid DNA at approximately 72° C. for approximately 15 seconds is repeated approximately 35 times. Then, the resulting plasmid DNA is extended again at approximately 72° C. for approximately 2 minutes, thereby obtaining the amplified plasmid DNA. This last additional extension is carried out by adding Cy5-dUTP.

Also, a hybridization reaction takes place on the aforementioned two DNA chips shown in FIGS. 2 and 3. The hybridization is performed at approximately 40° C. and, approximately 10 uL of a plasmid amplification product and approximately 5 uL of a globulin amplification product are mixed to be used as a reaction specimen. Approximately 10% by volume (i.e., volume-volume percentage (v/v)) of approximately 3 molar (M) aqueous sodium hydroxide (NaOH) is added to an electrical reaction specimen to cause a degeneration reaction at a room temperature for approximately 5 minutes. Then, approximately 5% by volume (i.e., volume-volume percentage (v/v)) of approximately 1 molar Tris-HCl whose pH is approximately 7.2 is added thereto to neutralize the above degeneration reaction. Afterwards, approximately 10% by volume (i.e., volume-volume percentage (v/v)) of approximately 3 molar hydrochloric acid (HCl) is added to the neutralized resulting product and then, the resulting product is placed into ice for approximately 5 minutes. A hybridization solution of 6×SSPE manufactured by Sigma, Co. is used to carry out the hybridization. Afterwards, a solution of 3×SSPE and a solution of 1×SSPE are respectively used to clean the DNA chips for approximately 2 minutes and then, the DNA chips are dried by a spin dryer.

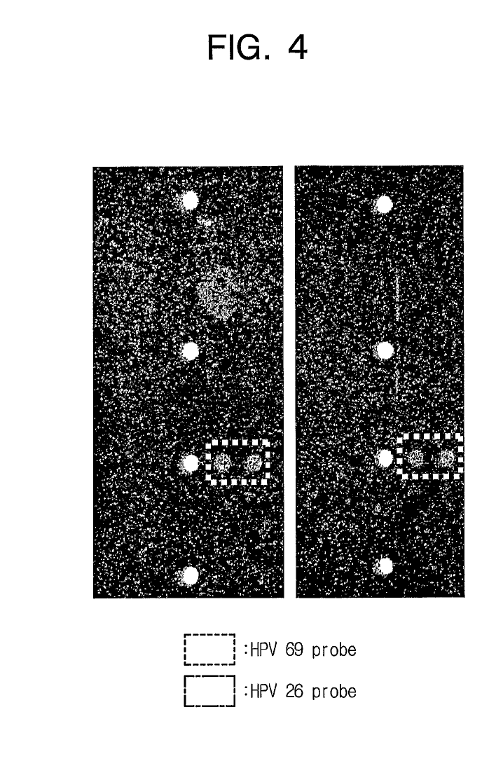

Fluorescent signals of the dried DNA chips are analyzed by using a confocal laser scanner where excitation occurs at approximately 650 nm and emission occurs at approximately 668 nm (GSI Luminomics, Germany). The fluorescence analysis result is shown in FIG. 4.

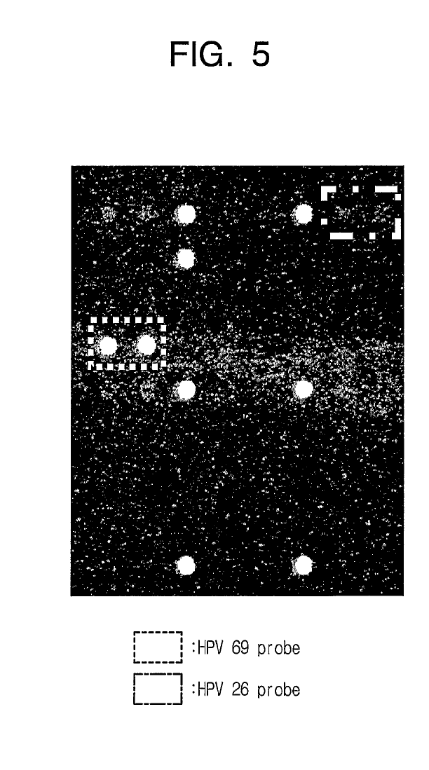

AS shown in FIG. 4, the comparison result on signals of the HPV 69 probe verifies that the HPV 69 probe according to the present invention exhibits higher specificity than the conventionally known HPV 69 probe. That is, in FIG. 4, the HPV 69 probe reacts with a HPV 26 probe of the specimen non-specifically, thereby expressing a strong signal level. On the other hand, in FIG. 5, the HPV 69 probe does rarely react with the HPV 26 probe of the specimen. Table 5 provided below shows the quantified signal levels.

TABLE 5

| SEQ ID No: | Signal to Background Ratio |
|---|---|
| 116 | 1.56 |
| 325 | 7.25 |

In Table 5, the signal to background ratio is obtained by dividing an average value of signals from individual probes by an average value of background signals.

### INDUSTRIAL APPLICABILITY

In accordance with embodiments of the present invention, it is possible to achieve a method of selecting a highly specific probe from a predetermined range of nucleotide sequences. On the basis of the highly specific probe selection method, such a probe that has high specificity allowing an annealing of the probe to DNA and RNA of HPV can be effectively selected. For instance, among nucleotide sequences of an L1 gene of HPV, such a probe having high specificity depending on genotypes of HPV can be effectively selected. Also, according to the present invention, those nucleotide sequences set forth in SEQ ID NOS: 1 to 286 can make a complementary base pair with DNA and RNA of HPV with high specificity.

Although the specific embodiments of the invention have been disclosed for illustrative purposes, those skilled in the art will appreciate that various modifications, additions and substitutions are possible, without departing from the scope and spirit of the invention as disclosed in the accompanying claims.

1. A method for selecting a highly specific probe among a predetermined range of nucleotide sequences, comprising:
setting a group of nucleotide sequences to be analyzed among the predetermined range of nucleotide sequences;
setting a range of nucleotide sequences of probes to be selected in the group of nucleotide sequences;
selecting first candidate probes whose length ranges from approximately 20 mer to approximately 50 mer within the range of nucleotide sequences of the probes;
selecting second candidate probes whose melting temperature with target nucleic acids ranges from approximately 50° C. to approximately 80° C. among the first candidate probes;
selecting third candidate probes whose melting temperature with nucleotide sequences except for the nucleotide sequences of the target nucleic acids among the group of nucleotide sequences is lower than a hybridization temperature among the second candidate probes; and
selecting fourth candidate probes among the third candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

2. The method of claim 1, wherein the first candidate probes selected within the range of nucleotide sequences of probes to be selected have a length ranging from approximately 30 mer to approximately 35 mer.

3. The method of claim 1, wherein the melting temperature between the target nucleic acids and the second probe candidates ranges from approximately 65° C. to approximately 75° C.

4. The method of claim 1, wherein the melting temperature of the third candidate probes and the nucleotide sequences except for the nucleotide sequences of the target nucleic acids is lower than the hybridization temperature by approximately 5° C. to approximately 10° C. and lower than a temperature that is lower than the hybridization temperature by approximately 5° C. to approximately 10° C.

5. The method of claim 1, wherein the melting temperature of the secondary structure of each fourth candidate probe is higher than the hybridization temperature.

6. A method for selecting a highly specific probe for a HPV genotype analysis, comprising:
selecting one or more nucleotide sequences selected from the group consisting of an L1 gene, an E6 gene and an E1 gene according to each HPV genotype; selecting first candidate probes whose length ranges from approximately 20 mer to approximately 50 mer within the nucleotide sequences;
selecting second candidate probes whose melting temperature with target nucleic acids ranges from approximately 50° C. to approximately 80° C. among the first candidate probes;
selecting third candidate probes whose melting temperature with nucleotide sequences except for the nucleotide sequences of the target nucleic acids among the nucleotide sequences is lower than a hybridization temperature among the second candidate probes; and
selecting fourth candidate probes among the third candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

7. The method of claim 6, wherein in each of the HPV genotypes, the nucleotide sequences of the L1 gene is selected.

8. The method of claim 6, wherein the first candidate probes selected within the nucleotide sequences have a length ranging from approximately 30 mer to approximately 35 mer.

9. The method of claim 6, wherein the melting temperature of the target nucleic acids and the second probe candidates ranges from approximately 65° C. to approximately 75° C.

10. The method of claim 6, wherein the melting temperature of the third candidate probes and the nucleotide sequences except for the nucleotide sequences of the target nucleic acids is lower than the hybridization temperature by approximately 5° C. to approximately 10° C. and lower than a temperature that is lower than the hybridization temperature by approximately 5° C. to approximately 10° C.

11. The method of claim 6, wherein the melting temperature of the secondary structure of each fourth candidate probe is higher than the hybridization temperature.

12. A method for selecting a highly specific probe for a HPV genotype analysis, wherein at least one pair is selected from a pair of SEQ ID NOS: 301 and 302 or a pair of SEQ ID NOS: 303 and 304 which are selected from the group consisting of primers for HPV and, a portion of a gene that is pertained to each HPV genotype and amplified by the selected primer pair is determined, the method comprising:
selecting first candidate probes whose length ranges from approximately 20 mer to approximately 50 mer within the portion of the gene;
selecting second candidate probes whose melting temperature with target nucleic acids ranges from approximately 50° C. to approximately 80° C. among the first candidate probes;
selecting third candidate probes whose melting temperature with nucleotide sequences except for nucleotide sequences of the target nucleic acids among a group of nucleotide sequences to be analyzed is lower than a hybridization temperature among the second candidate probes; and
selecting fourth candidate probes among the third candidate probes, wherein a secondary structure of each fourth candidate probe has a melting temperature lower than the hybridization temperature by approximately 5° C. to approximately 10° C. and higher than a temperature that is lower than the hybridization temperature by approximately 5° C. to approximately 10° C.

13. The method of claim 12, wherein the first candidate probes selected within the selected group of nucleotide sequences to be analyzed have a length ranging from approximately 30 mer to approximately 35 mer.

14. The method of claim 12, wherein the melting temperature of the target nucleic acids and the second probe candidates ranges from approximately 65° C. to approximately 75° C.

15. The method of claim 12, wherein the melting temperature of the third candidate probes and the nucleotide sequences except for the nucleotide sequences of the target nucleic acids is lower than the hybridization temperature by approximately 5° C. to approximately 10° C. and lower than a temperature that is lower than the hybridization temperature by approximately 5° C. to approximately 10° C.

16. The method of claim 12, wherein the melting temperature of the secondary structure of each fourth candidate probe is higher than the hybridization temperature.

17. A probe that is complementary with DNA and RNA of HPV, wherein the probe is selected from the group consisting of oligonucleotides having nucleotide sequences of SEQ ID NOS: 1 to 286 by employing the method according to claim 1.

18. The probe of claim 17, wherein the probe is selected from the group consisting of
oligonucleotides having nucleotide sequences of SEQ ID NOS: 1, 7, 14, 26, 27, 34, 41, 50, 54, 59, 61, 66, 75, 80, 83, 89, 97, 109, 113, 125, 140, 151, 166, 172, 184, 189, 207, 213, 217, 228, 236, 249, 264, 270, 276, and 283.

19. The probe of claim 18, wherein the probe is selected from the group consisting of
oligonucleotides having nucleotide sequences of SEQ ID NOS: 1, 7, 26, 27, 34, 41, 50, 54, 59, 61, 66, 75, 80, 83, 89, 97, 109, 113, 125, and 140.

20. A DNA chip for HPV including at least one probe according to claim 17.
